(19) United States
(12) Patent Application Publication
(10) Pub. No.: US 2019/0279404 A1
(43) Pub. Date: Sep. 12, 2019

(54) METHODS AND PROGRAM PRODUCT FOR MAPPING OF FUNCTIONAL PANELS ONTO AVAILABLE PHYSICAL DISPLAYS

(71) Applicant: Jim Schwaiger, Huron, SD (US)

(72) Inventor: Jim Schwaiger, Huron, SD (US)

(21) Appl. No.: 15/917,792

(22) Filed: Mar. 11, 2018

(51) Int. Cl. G06T 11/00; G16H 30/20

(52) U.S. Cl. CPC G06T 11/008; G06F 3/04845; G16H 30/20

(57) ABSTRACT

A method includes retrieving session data for a radiologist. The session data including viewing preferences for the radiologist. The radiologist is utilizing a computing device for viewing images for a session. Two functional panels associated with the session are accessed. The two functional panels are mapped to display devices in communication with the computing device. The mapping uses the viewing preference and configurations of the display devices. The mapping is communicated to the display devices for viewing by the radiologist.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to mapping of functional panels onto available physical displays. More particularly, certain embodiments of the invention relates to mapping medical images onto imaging stations.

It is believed that current state of the art involves radiologists, or support staff, may largely hand craft image layouts for a specific workstation, such as, but not limited to, number, size and shape of monitors, being used by the radiologist. Many details including, without limitation, the size, shape and number of images being displayed on a monitor are adjustable. Typically these details may be set for each exam type. Typically these may be hand crafted layouts designed for each individual view station configuration.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following are examples of a specific aspects in the prior art that, while expected to be helpful to further educate the reader as to additional aspects of the prior art, are not to be construed as limiting the present invention, or any embodiments thereof, to anything stated or implied therein or inferred thereupon. Some systems recognize that radiologists often compare a current exam to previous exam(s), and seeks the best direct visual comparison. This includes side by side display of Primary and Comparison images/series, synchronized so that user can scroll the Primary and Comparison scrolls automatically for image by image comparison.

By way of educational background, other aspects of the prior art generally useful to be aware of is that dozens of different exam types multiplied by several different display configurations creates a huge number of layouts for each radiologist. To reduce the size of this challenge, some methods observe, record and analyze behavior and preferences of radiologists as they review studies in hope of learning enough to infer their preferences for exam types they have not yet configured.

By way of educational background, other aspects of the prior art generally useful to be aware of is some methods address specific hardware challenges for mixed monitor displays.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIGS. 1a-1i, illustrate exemplary panel placement on a user interface, in accordance with embodiments of the present invention;

FIGS. 2a-2h, illustrate exemplary panel placement on a monitor configuration, in accordance with embodiments of the present invention;

FIG. 3 illustrates exemplary panel placement with extrinsic logical panels on a monitor configuration, in accordance with an embodiment of the present invention;

FIG. 4 illustrates exemplary panel placement with patient data panels on a monitor configuration, in accordance with an embodiment of the present invention;

FIG. 5 illustrates exemplary panel placement with patient data panels on a monitor configuration, in accordance with an embodiment of the present invention;

FIG. 6 illustrates an exemplary VRC system, in accordance with an embodiment of the present invention;

FIG. 7 illustrates an exemplary setup method, in accordance with an embodiment of the present invention;

FIG. 8 illustrates an exemplary mapping method, in accordance with an embodiment of the present invention;

FIG. 9 illustrates an exemplary GUI method, in accordance with an embodiment of the present invention; and

FIG. 10 is a block diagram depicting an exemplary client/server system which may be used by an exemplary web-enabled/networked embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settled law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognize in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of Claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising.” This term is open-ended. As used in the appended claims, this term does not foreclose additional structure or steps. Consider a claim that recites: “A memory controller comprising a system cache. . . . ” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. .sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s).

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

“Software” may refer to prescribed rules to operate a computer. Examples of software may include: code segments in one or more computer-readable languages; graphical and or/textual instructions; applets; pre-compiled code; interpreted code; compiled code; and computer programs.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hyper text Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., website owners or operators) place multimedia information (e.g., text, graphics, audio, video, animation, and other forms of data) at specific locations on the Internet referred to as webpages. Websites comprise a collection of connected, or otherwise related, webpages. The combination of all the websites and their corresponding webpages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

As used herein, the “client-side” application should be broadly construed to refer to an application, a page associated with that application, or some other resource or function invoked by a client-side request to the application. A “browser” as used herein is not intended to refer to any specific browser (e.g., Internet Explorer, Safari, FireFox, or the like), but should be broadly construed to refer to any client-side rendering engine that can access and display Internet-accessible resources. A “rich” client typically refers to a non-HTTP based client-side application, such as an SSH or CFIS client. Further, while typically the client-server interactions occur using HTTP, this is not a limitation either. The client server interaction may be formatted to conform to the Simple Object Access Protocol (SOAP) and travel over HTTP (over the public Internet), FTP, or any other reliable transport mechanism (such as IBM® MQSeries® technologies and CORBA, for transport over an enterprise intranet) may be used. Any application or functionality described herein may be implemented as native code, by providing hooks into another application, by facilitating use of the mechanism as a plug-in, by linking to the mechanism, and the like.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Additionally, the phrase “configured to” or “operable for” can include generic structure (e.g., generic circuitry) that is manipulated by software and/or firmware (e.g., an FPGA or a general-purpose processor executing software) to operate in a manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

While a non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; the non-transitory computer readable medium, however, does not include a pure transitory signal per se; i.e., where the medium itself is transitory.

As used herein, the term “radiologist” refers to a professional who works with medical images. Others professional who may also work with medical images include, but not limited to, neurologists, surgeons, chiropractors, veterinarians, dentists, etc.

As used herein, “radiologist” is inclusive of those using medical imaging stations for review or interpretation. As used herein, Exam is short for Examination, a common term referring to diagnostic imaging study performed on a patient. The words Exam and Imaging study are commonly interchanged herein and among radiologists.

Many embodiments, and variations thereof, may define a radiologist's interaction with an abstract functional panel using display independent parameters. In many embodiments, and variations thereof, parameters may allow a method to determine how and when the panels may be displayed for the radiologist on available displays. In many embodiments, a Virtual Radiology Canvas (VRC) may use two or more abstract functional panels which may have undefined size and variable position. Once the radiologist has expressed some simple preferences about how he wants to use these functional panels, the VRC method may manage the details of mapping the functional panels onto whatever physical display(s) the radiologist may be currently using. Some embodiments, may use three functional panels. In some embodiments, a Primary (P) Panel may display information related to a Primary set of images to be interpreted, a Comparison (C) Panel may display information for Comparison studies such as, but not limited to, often older versions of the same type of study, and a Selection (S) Panel may display information on exams available for Selection (viewing). In some other alternate embodiments, the S panel may be incorporated into the P or C panel. In some embodiments, additional panels may also be included in the system, including, but not limited to, a panel for handling an interactive imaging workstation, a panel for displaying patient information including vital signs such as, but not limited to, EKG, blood pressure, etc., a panel for technical information about the exam including contrast and radiation doses.

In many embodiments, and variations thereof, the VRC may define when and how the functional panels are displayed to the radiologist (on multiple different physical display configurations) using one or more attributes/parameters.

In some embodiments, attribute(s) which determine how functional panels are mapped onto as physical displays as a Display List. A non-limiting example of how a Display List may work may be a set of lines, in the format: Line#:ABCXYZ, Line#:ABCXYZ, where the letters indicate the Panel type to be displayed. A single letter appearing more than once in a line indicates that more than one monitor is allocated to this panel.

The following are non-limiting examples of display lists:

1:PC indicates on a single display (1:), Primary and Comparison panels will be displayed;

1:P indicates on a single display (1:), only Primary panel is displayed;

1:PCS indicates on a single display (1:), Primary, Secondary and Selection panels will all be displayed;

2:PP indicates on a two display system (2:), both displays (P & P) will be used for the Primary panel;

2:PC indicates on a two display system (2:), one display will be used for P-Primary and one for C-Comparison Panels;

3:PPS indicates on a three display system (3:), two displays are used for P-Primary Panel and one for S-Selection Panel; and

3:PCS indicates on a three display system (3:), one display is used for P-Primary, one for C-Comparison and one for S-Selection Panels. The non-limiting examples of display lists only showed a limited number of display systems and a limited number of panels. One of ordinary skill may readily recognize that the number of display systems and panels may not be limited.

Display list may indicate which panels are displayed preferentially with variable numbers of monitors. It is believed that for many radiologists, simply setting this attribute to their preference may resolve about 80%+ of their needs, with fine tuning using other attributes. In other embodiments, other schemes such as, but not limited to, Display Profiles may be utilized.

In some embodiments, panel attributes may fall into two broad classes, relational and intrinsic. Relational attributes may affect how the panels relate to each other, including, without limitation, how and when panels are displayed. Intrinsic attributes may affect the panel itself, including, without limitation, the format of images and other information displayed within, allowing various customized styles of interaction with the radiologist.

Non-limiting relational attribute examples for the functional panels are listed below. It is believed that many radiologists may be quite happy with the default settings in most cases, but the attributes may allow for high levels of customization when desired.

Multiple—NONE if no preference

Yes—can use multiple monitors

No

Geometry—NONE if no preference

Horizontal

Vertical

Resolution—NONE if no preference, Default is “Mid” for Primary/Comparison Panels

Low—typically used for text displays such as Exam Selection

NotLow(Mid)—specifies non text display, can use a high resolution display

High—requires high resolution (e.g. Primary/Comparison imaging Panels)

Sharing—NONE if no preference, typical default is NO for Primary

Yes—may share a logical display with another panel

No—desires exclusive use of display (typically for Primary Panel)

Splittable—NONE if no preference

Yes—allows panel to split across two physical displays

No—does not allow panel to be split across two displays, although panel may use multiple displays in their entirety

Vertical Shrink—NONE if no preference

Yes—allows vertical only shrink by up to 50%

No—does not allow vertical shrink (not preserving aspect ratio)

HorizontalShrink—NONE if no preference

Yes—allows horizontal shrink by up to 50%

No—does not allow horizontal shrink (not preserving aspect ratio)

Altitude—NONE if no preference

Top

Mid

Bottom

Color—NONE if no preference

Yes—requires color

No—does not have color

Lateral—NONE if no preference

Left

Mid

Right

Size—NONE if no preference

Small—often used by Exam Selection panel

Mid

High—requires high resolution if present

Non-limiting Intrinsic attribute examples are listed below:

Windows # of windows displayed in the panel. Common examples include:
1—the entire panel is used to display a single window/image
2—panel is split into two windows
3 . . . 6—the panel into a number of windows approximating the target number, based on geometry of display

Scrollable—NONE if no preference

Yes—Images for a study can be scrolled (PA & lateral chest displays PA on

Full screen and allows scrolling/bar selection of other views

Synchronized—NONE if no preference

Yes—synchronizes scrolling between Primary and Comparison Panels

No—prefers individual scrolling of Primary/Comparison Panels

Display Priorities NONE (ordinal) if no preference
Cross sectional exams often have several series, and a radiologist may prefer that certain series be preferentially displayed and others available for review. E.g. the radiologist may prefer a 2 window panel with the axial images and coronal images displayed in the two windows, and other images would be available for selection if desired.

ControlBarLocation NONE if no preference

Top—of panel

Bottom

Left

Right

ControlTextSizeNONE if no preference, else specify font size

ControlTextFontNONE if no preference

ControlTextColor NONE if no preference

ImageTextSize NONE if no preference

ImageTextFont NONE if no preference

ImageTextColorNONE if no preference

In some embodiments, the attributes outlined above may be defined at multiple levels: Global (defined by manufacturer), System (defined by administrator of the Picture Archiving And Communication System (PACS)/Hospital Network), Group (Clinics), User (Radiologists), Panel Type {Primary, Comparison, Selection}, {Imaging} Class (plain films, Cross Sectional, Nuclear, Mammography), {Imaging} Modality (CT, US, MRI, etc.), and Exam Type (defined by PACS/Hospital Network administrator). The levels to the left are higher in scope and range, the levels to the right are higher in specificity, and override the levels to the left. This may allow broad defaults to be set at the upper (left) levels, and radiologist or exam based preference to override them toward the right.

In some embodiments, placing the radiologist attributes as the most specific may allow the radiologist to override any of the attributes set at the higher levels, as the radiologist deems appropriate for their needs. Often the radiologist overriding preferences may be fairly minimal, limited to the Display List and Top/Left preferences.

In many embodiments, the Virtual Radiology Canvas may allow a radiologist to design their interaction with functional panels, which may be divorced from the physical displays available on any given system. In many embodiments, the functional panels may allow the radiologist's interaction to be intuitive and familiar, even as the number, size and shape of available physical monitors may change. In many embodiments, the functional panels have one or more relational attributes defining how the panels relate to each other, and may allow the method to map these functional panels onto physical displays. In many embodiments, the functional panels have one or more intrinsic attributes which define how information (including images) may be displayed within the panels, and how the radiologist interacts with the panels. In many embodiments, the functional panels may allow the radiologist to efficiently define their general preferences, independent of display number and resolution, and these general preferences may allow their style of interaction to be essentially consistent across most/all of the different imaging studies he views.

In some embodiments, because preference sets are display independent, they may be created or collected by manufacturers and published for use by radiologists. Radiologists may start with these designed sets and fine tune them by answering a few simple questions about their preferences. This may dramatically improves the configuration process. In some embodiments, a pathologist may use VRC to present radiology images combined with, but not limited to, microscopic images and clinical data for a multispecialty tumor board. This non-limiting example illustrates how VRC may be used to integrate other data to meet the needs of other specialties, yet interaction with the panels is familiar to the pathologist even as he moves across different display systems. In some embodiments, Internist/Intensive Care Unit caring for critically ill patients may often require integrating medical imaging with other information sources, including, patient monitors such as, but not limited to, vital signs, EKG, respirators, etc. VRC may allow integration of these external sources with the imaging in an intuitive way, honoring the display independent preferences of the professionals involved. In some embodiments, VRC may be used to integrate medical imaging with other images and data such as EEG, EMG, sleep studies, laboratory results used by Neurologists.

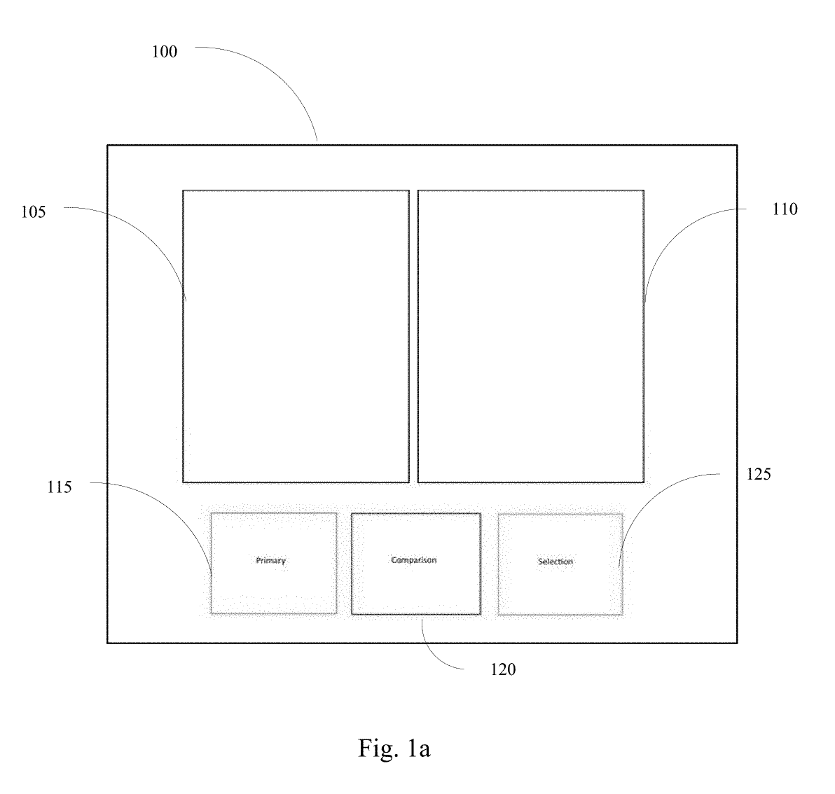

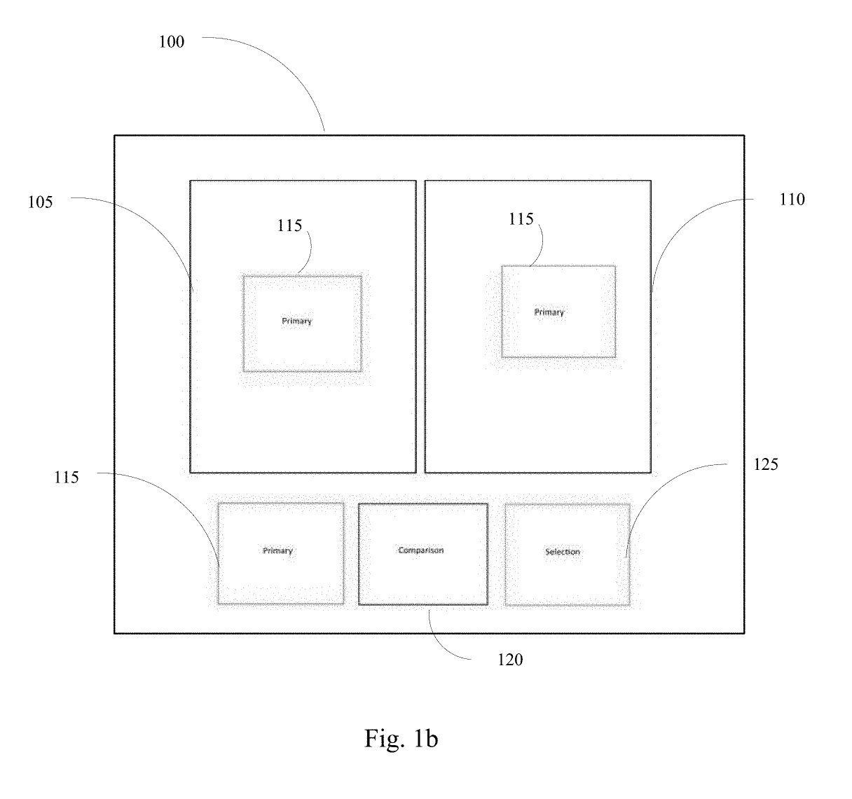

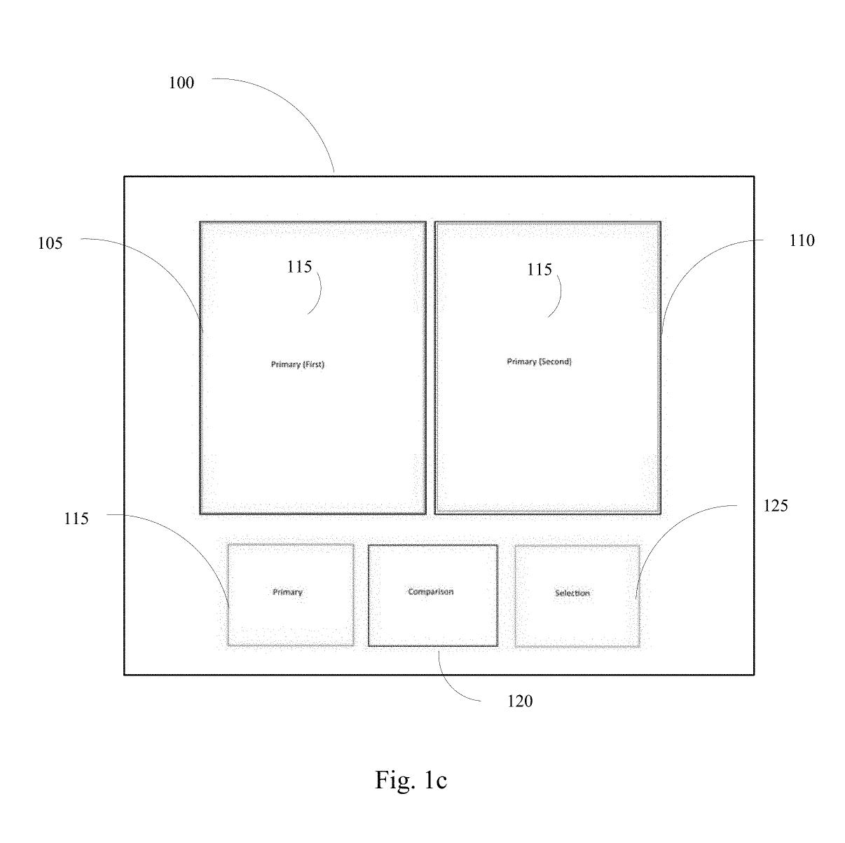

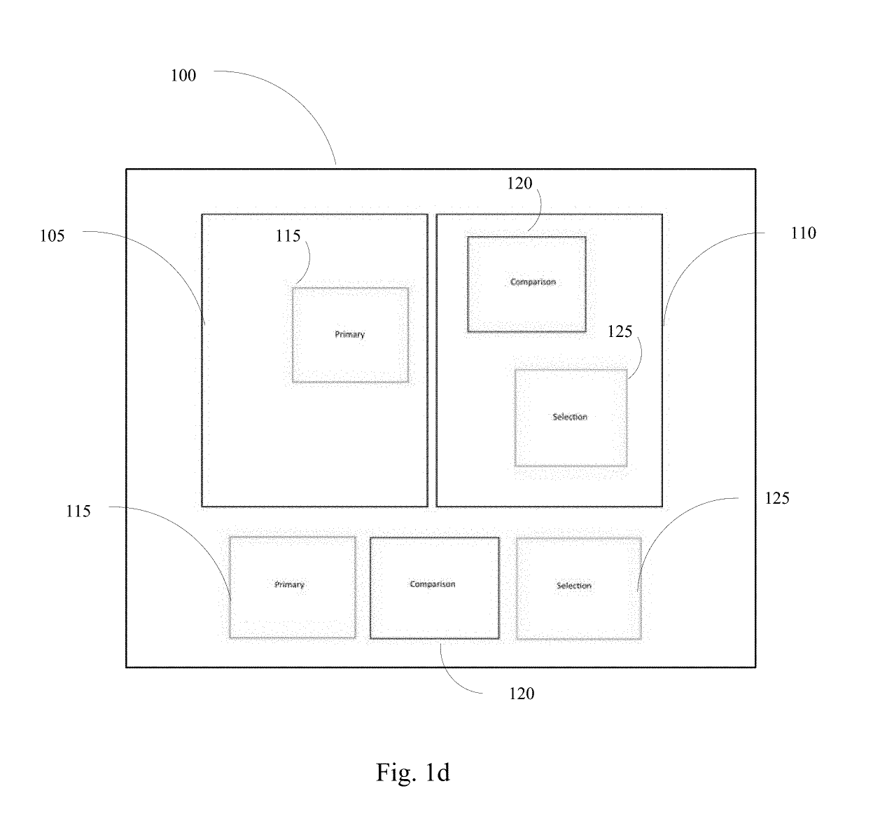

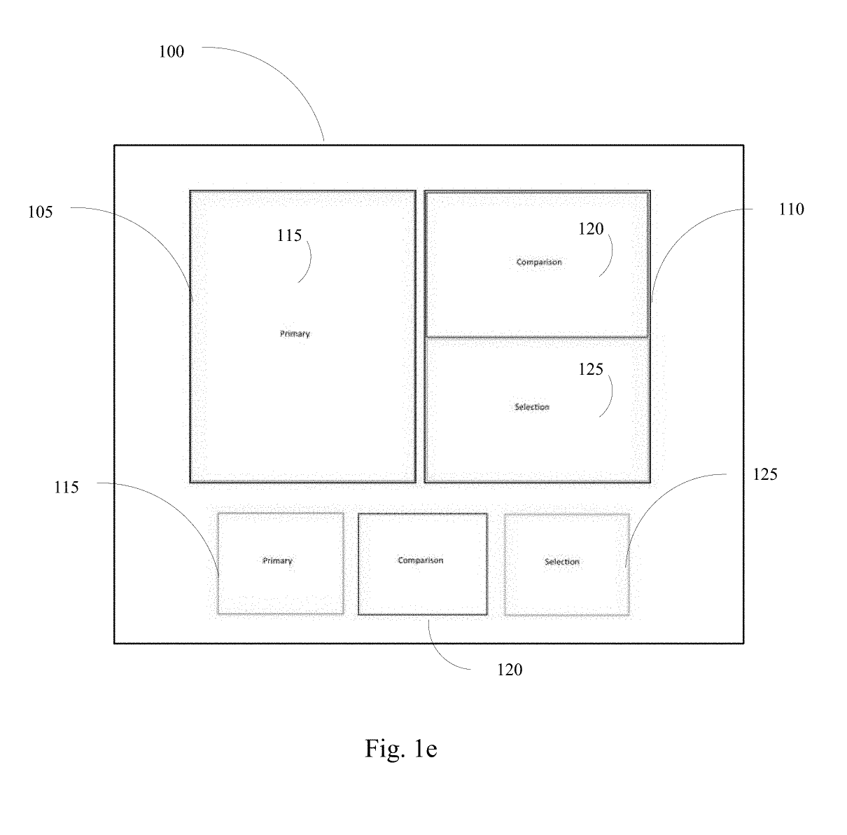

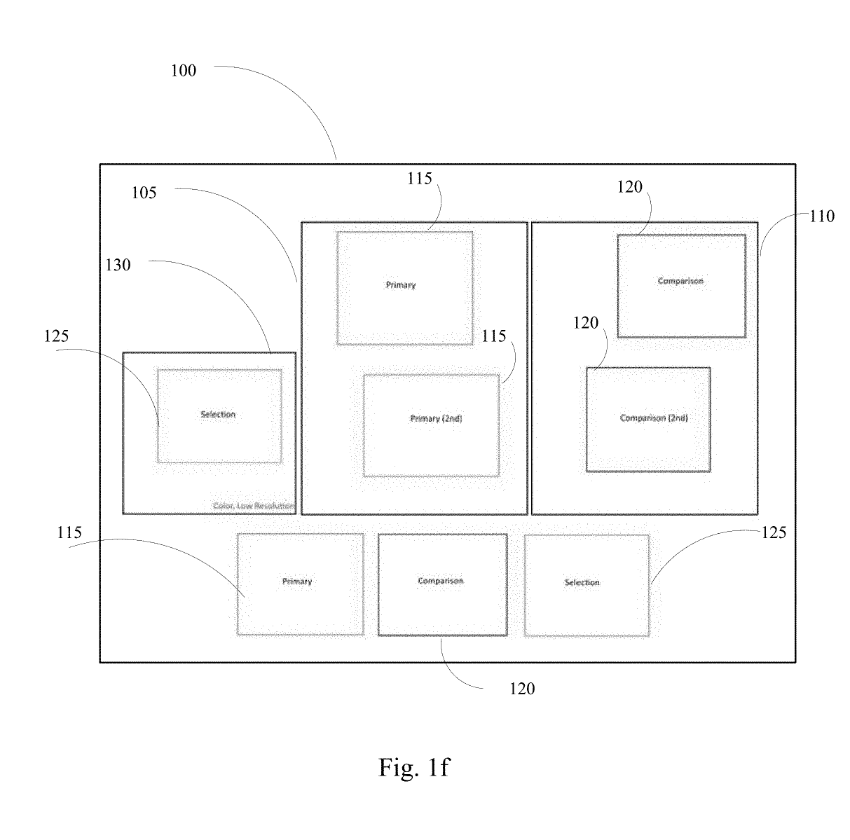

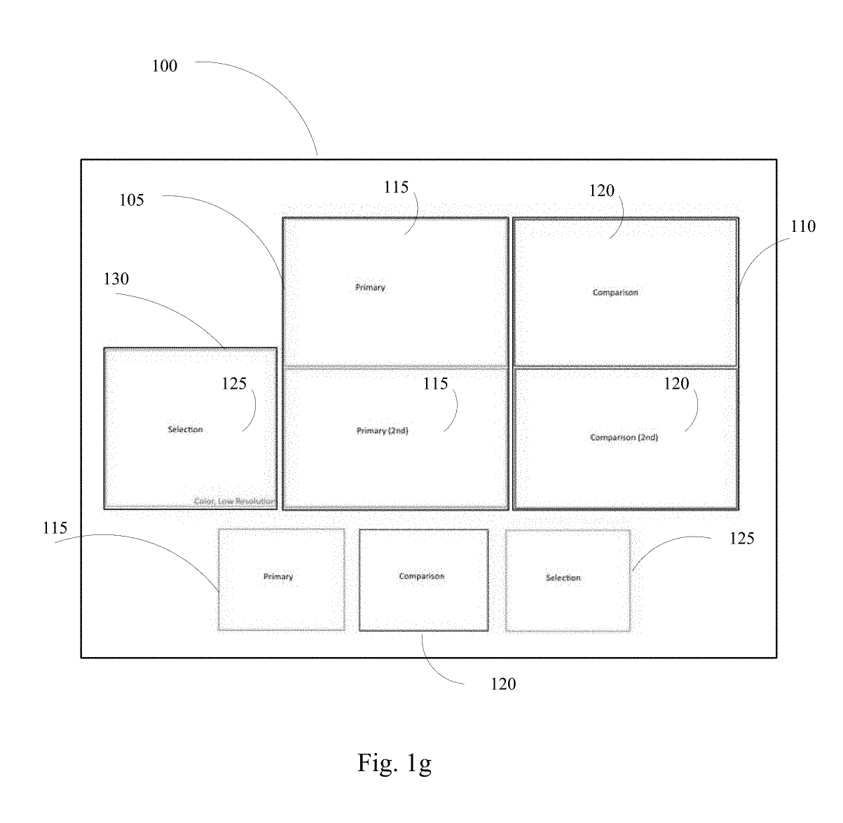

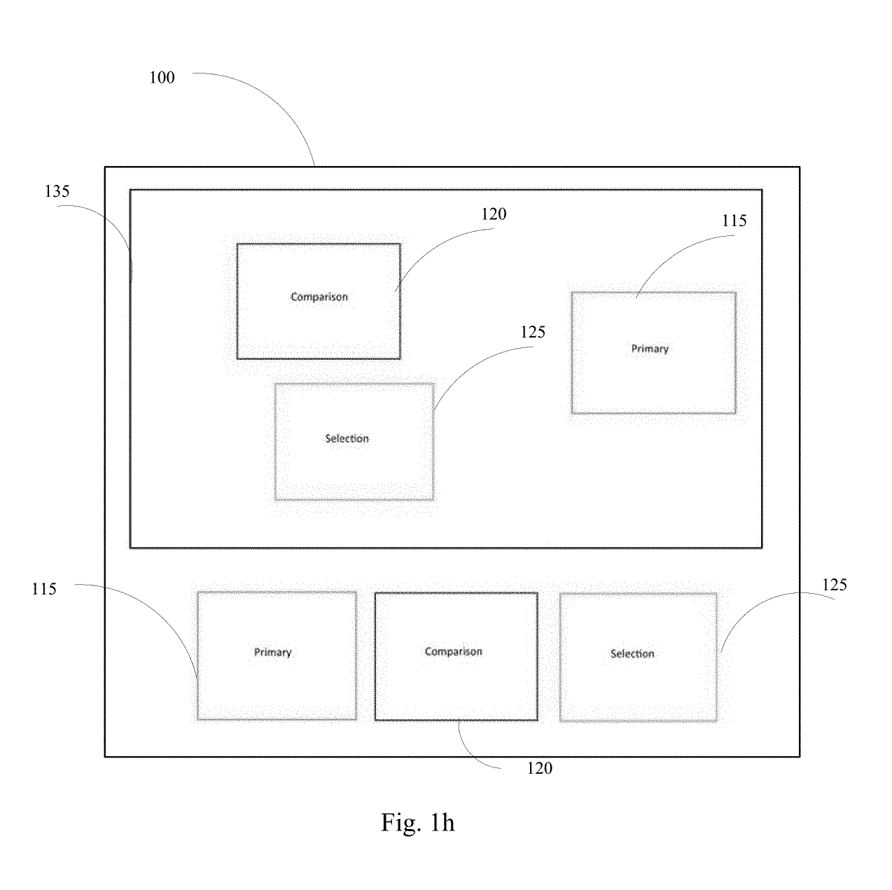

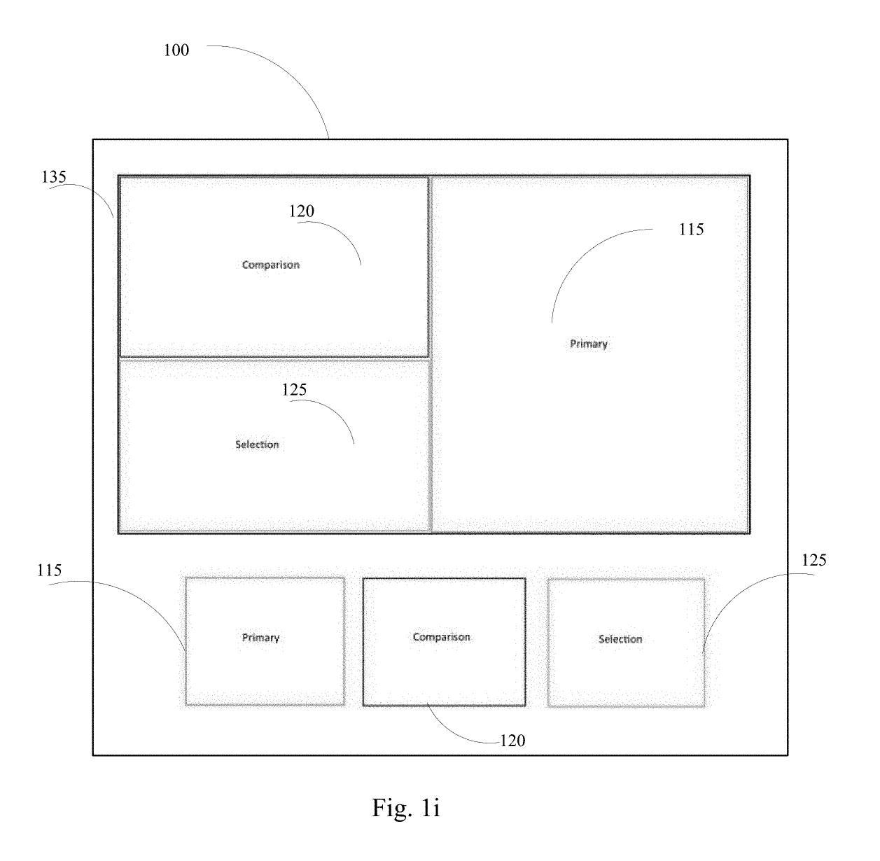

FIGS. 1a-1i, illustrate exemplary panel placement on a user interface, in accordance with embodiments of the present invention. In the present embodiments, display profiles may allow the radiologist to specify the relational position of panels in different display configurations. These relational positions may be stored as attributes to allow positioning of panels on similar display configurations encountered in the future. A radiologist wishing to designate panel display preferences for a given type of display configuration may be presented with a corresponding graphic use interface (GUI) screen 100. In the present embodiments, GUI screen 100 may be controlled as part of a VRC setup program where the radiologist's preferences are obtained and stored. Referring to FIG. 1a, the radiologist may be presented with a thematic screen 100 depicting two vertical monitors 105 and 110 (of unknown resolution and dimensions) and a primary panel 115, a comparison panel 120 and a selection panel 125. If the radiologist wants to focus on viewing just the primary panel, the radiologist may configure it as shown in FIG. 1b. In this figure, the radiologist has placed a copy of primary panel 115 on vertical display 105 and vertical display 110. The radiologist may use various methods to accomplish this depending on the monitor/computer being used such as, but not limited to, click and drag a mouse pointer, touch and drag for touch panel display, keyboard entry, etc. The radiologist does not need to place these panels precisely in the symbolic displays 105 and 110, since their size and dimension is not known. The radiologist may then click on a “Done” button (not shown), and the panels are automatically displayed as they might appear on a dual monitor configuration shown in FIG. 1c. In the present embodiment, the “Done” button may be an area on screen 100, a keyboard entry, a mouse control, etc. Referring to FIG. 1d, the radiologist may have desired to view all three panels 115, 120, and 125 and placed them as shown. The radiologist may then click on the “Done” and the panels are automatically displayed as they might appear on a dual monitor configuration shown in FIG. 1e. Referring to FIG. 1f, the radiologist may have desired to view all three panels 115, 120, and 125 and placed them as shown on vertical monitors 105 and 110 and low resolution monitor 130. The radiologist may then click on the “Done” and the panels are automatically displayed as they might appear on vertical monitors 105 and 110 and low resolution monitor 130 shown in FIG. 1g. Referring to FIG. 1h, the radiologist may have desired to view all three panels 115, 120, and 125 and placed them as shown on horizontal monitor 135. The radiologist may then click on the “Done” and the panels are automatically displayed as they might appear on horizontal monitor 135 shown in FIG. 1i. One of ordinary skill may readily recognize that other monitor configurations may be displayed and other or additional panels may be displayed. This is a simple and elegant approach to designing panel placement for different display configurations. In other embodiments the side of a panel may be dragged to give a smaller or larger portion of the screen to that panel, rather than the 50% which is used as the default. In other alternative embodiments, the order or placement of the monitors may be changed by moving/dragging a monitor on screen 100 such as, but not limited to, moving low resolution monitor 130 to a different position where the radiologist may expect to view it at. In the present embodiments, if the radiologist encounters a new combination, it is anticipated that moving the panels into reasonable placement may take less than a minute. In the present embodiments, the preferences for this Display Profile may not be stored as absolute numbers, but in terms of, without limitation, relative positions, fractions of that side of the display, etc. so they may be translated to a similar presentation with different specific resolutions.

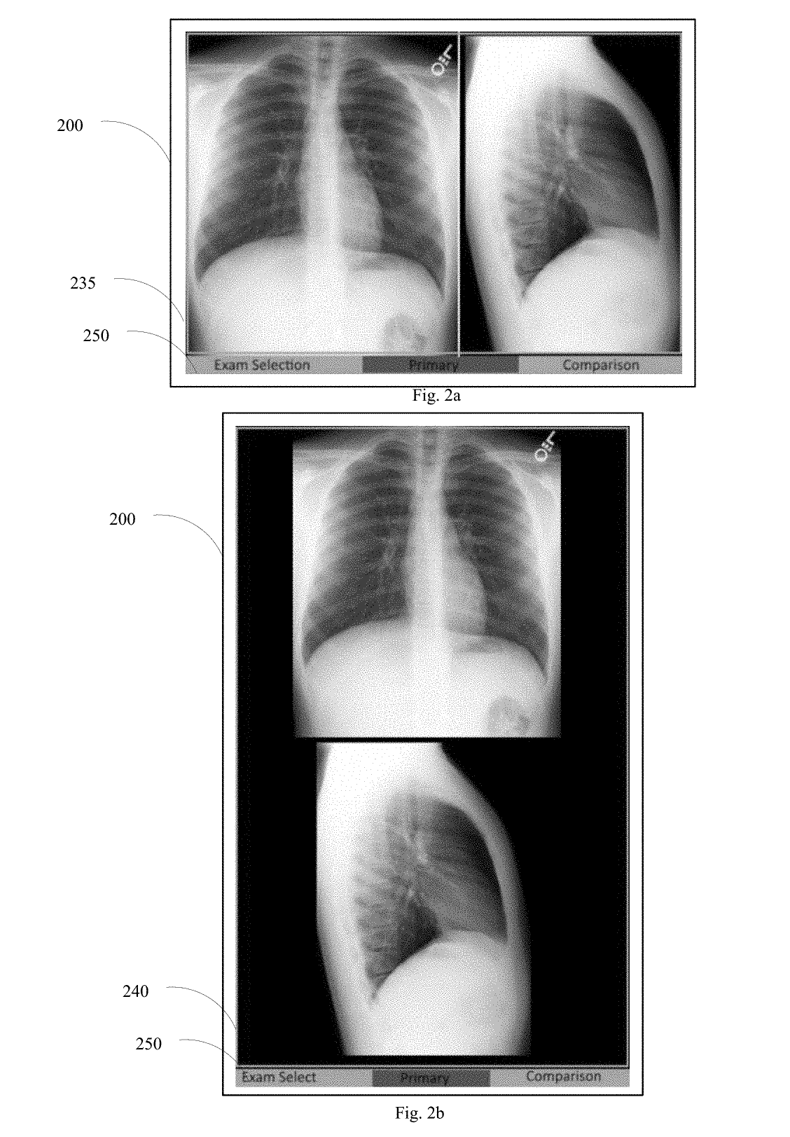

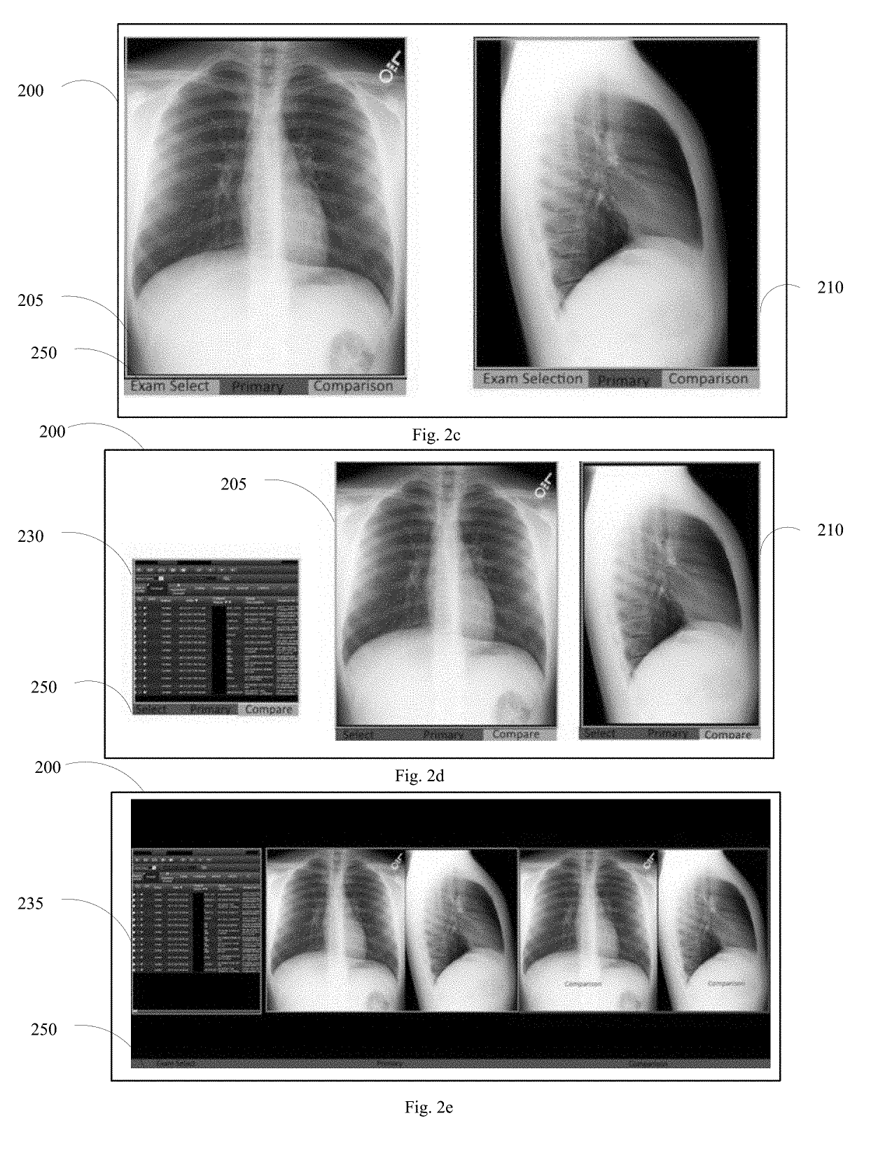

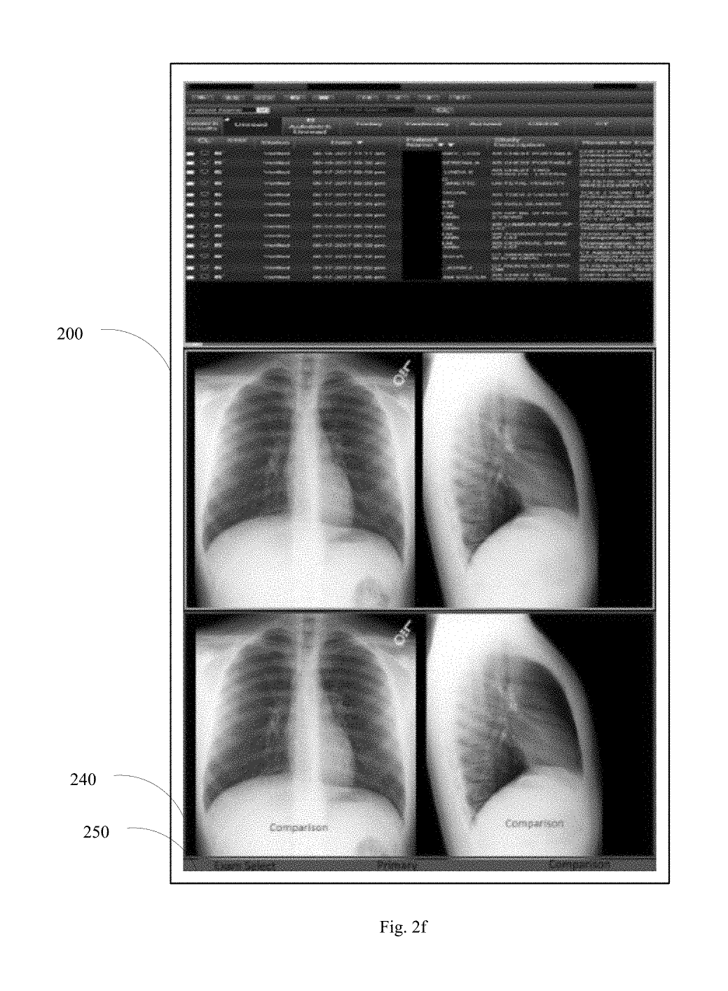

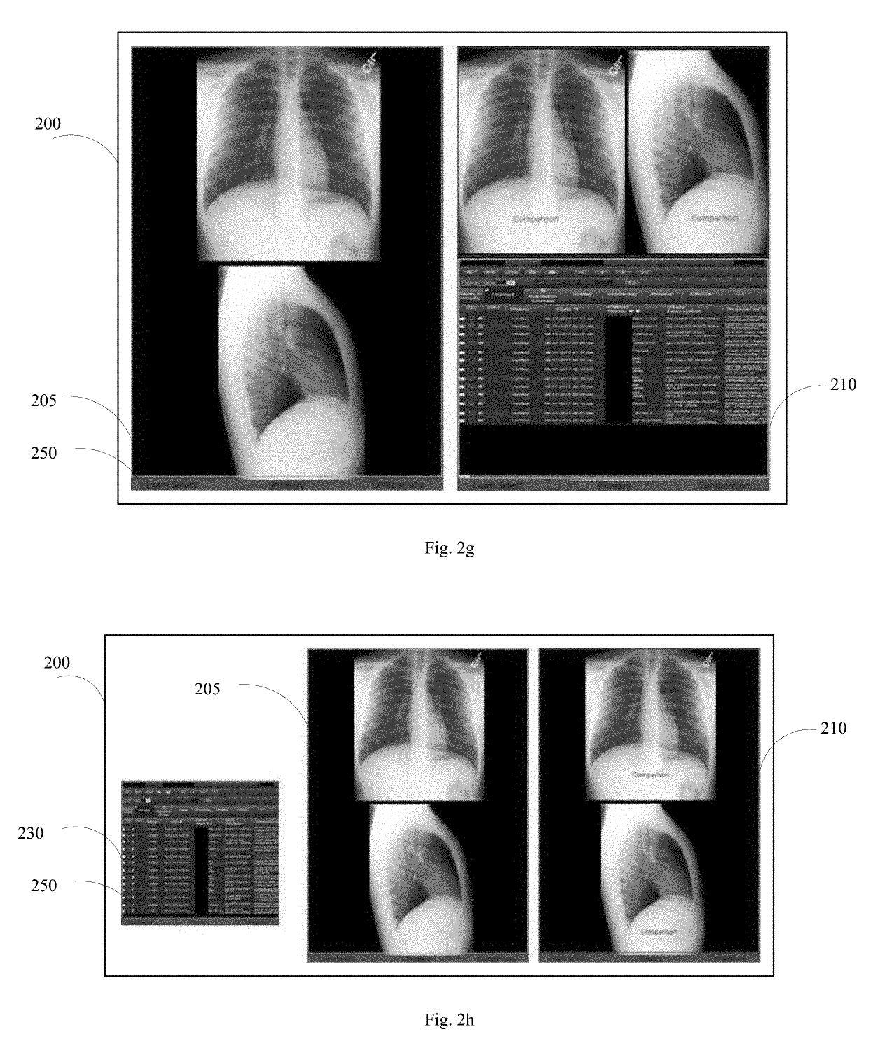

FIGS. 2a-2h, illustrate exemplary panel placement on a monitor configuration, in accordance with embodiments of the present invention. In the present embodiments, a first radiologist may have set their Display List to “1:P,2:PP,3:PPCS, 4:PPCS”. Referring to FIG. 2a, the first radiologist may have selected to view an exam on a horizontally oriented display 235 such as, but not limited to, a tablet computer. Since the first radiologist has set a 1:P in their display list primary exam images may be initially displayed. A navigation section 250 may allow the first radiologist to view the Exam Selection or the Comparison. Referring to FIG. 2b, the first radiologist may have selected to view the exam on a vertically oriented display 240 such as, but not limited to, a tablet computer. Since the first radiologist has set a 1:P in their display list primary exam images may be initially displayed. A navigation section 250 may allow the first radiologist to view the Exam Selection or the Comparison. Referring to FIG. 2c, the first radiologist may have selected to view the exam on two vertical displays 205 and 210 such as, but not limited to, a viewing station in a hospital. Since the first radiologist has set a 2:PP in their display list, primary exam images may be initially displayed on vertical displays 205 and 210. A navigation section 250 may allow the first radiologist to view the Exam Selection or the Comparison. Referring to FIG. 2d, the first radiologist may have selected to view the exam on a low resolution monitor 230 and two high resolution vertical displays 205 and 210 such as, but not limited to, a viewing station. Since the first radiologist has set a 3:PPCS in their display list, primary exam images may be initially displayed high resolution vertical displays 205 and 210. In the present embodiment, both high resolution imaging monitors have been devoted to the primary panel, honoring first radiologist's wish to use get the best possible view of the current study. The first radiologist may select the Comparison panel if he wishes to check for changes. The smaller low resolution monitor 230 to the left has not been claimed for the primary panel, because the default resolution attributes for the primary and comparison panels may have been set to “Mid”. VRC therefore may not use the small (low resolution) monitor 230 for imaging, but rather uses it to display the Exam Selection list. A navigation section 250 may allow the first radiologist to view the Exam Selection or the Comparison.

In the present embodiments, a second radiologist may have set his attributes the same as the first radiologist, differing only in his settings of the Display List attribute, which may determine the order in which panels may be displayed. The second radiologist may want to be sure he sees everything at the same time. His Display List may be set to “1:PCS”, “2:PCS”, “3:PCS”, “4:PCSP”. Referring to FIG. 2e, the second radiologist may have selected to view an exam on a horizontally oriented display 235 such as, but not limited to, a tablet computer. Since the second radiologist has set a 1:PCS in their display list, primary exam images, comparison images and selection panel may be initially displayed In some embodiments, PCS may designate which panels may be displayed, but may not specify order/position. In some embodiments, the position may be determined by the relational attributes, some of which may have their default values, and some which may be set by the user to control positioning. In a non-limiting example, by default, the Resolution parameter of the Selection panel may be set to LOW, indicating that it may preferentially be placed on lower resolution displays. The default value of Resolution for Primary and Comparison panels may be Medium or High, indicating they may preferentially be placed on high(er) resolution displays to maximize image quality. Using only these default values for Resolution, the Selection panel may be automatically placed on the lower resolution display. In the example image, it depicts the left most display as having a different shape (perhaps smaller) than the two identical high resolution image displays to the right. This may often be the pattern in radiology departments—text displays are smaller than the imaging displays. Additional fine tuning may be accomplished by setting or using the default values of positional attributes for the panels (e.g. the Horizontal attribute for Primary may be set to LEFT (its default value) if the radiologist prefers that Primary images be displayed to the left). The size and shape of the tablet computer may limit the configuration by VRC, leaving a fair bit of wasted space. Nonetheless, this display honors the second radiologist's preference to see all panels by default. If he wishes to see any image in more detail, he may double click on it to use the entire screen, and double click again to toggle back. Referring to FIG. 2f, the second radiologist may have selected to view an exam on a vertically oriented display 240 such as, but not limited to, a tablet computer. Since the second radiologist has set a 1:PCS in their display list, primary exam images, comparison images and selection panel may be initially displayed. In some embodiments, PCS may not dictate the order/position of the panels. In the present embodiment, the geometry of the tablet may allow the vertical configuration to make better use of available space. Referring to FIG. 2g, the second radiologist may have selected to view the exam on two vertical displays 205 and 210 such as, but not limited to, a viewing station in a hospital. Since the second radiologist has set a 2:PCS in their display list, primary exam images may be initially displayed on vertical display 205 and comparison images on vertical display 210 along with the selection panel. A navigation section 250 may allow the first radiologist to view the Exam Selection or the Comparison. Referring to FIG. 2h, the second radiologist may have selected to view the exam on a low resolution monitor 230 and two high resolution vertical displays 205 and 210 such as, but not limited to, a viewing station. Since the second radiologist has set a 3:PCS in their display list, primary exam images may be initially displayed high resolution vertical display 205 and comparison images on high resolution display 210. In the present embodiment, both high resolution imaging monitors have been devoted to the primary panel and comparison panel. The smaller low resolution monitor 230 to the left has not been claimed for the primary or comparison panel, because the default resolution attributes for the primary and comparison panels may have been set to “Mid”. VRC therefore may not use the small (low resolution) monitor 230 for imaging, but rather uses it to display the Exam Selection list. A navigation section 250 may allow the second radiologist to view the Primary or the Comparison.

In some embodiments, it is very common for radiologists to prefer a scrolling configuration that may not display both views of a chest at the same time, but rather starts with the frontal view and allows the radiologist to scroll to the lateral (side) view. In some embodiments, panel attributes may fall into two broad classes, relational and intrinsic. Relational attributes may affect how the panels relate to each other, including, without limitation, how when panels are displayed. Intrinsic attributes may affect the panel itself, including, without limitation, the format of images and other information displayed within, allowing various customized styles of interaction with the radiologist. In some alternate embodiments, a Virtual Reality (VR) or augmented VR display may be utilized.

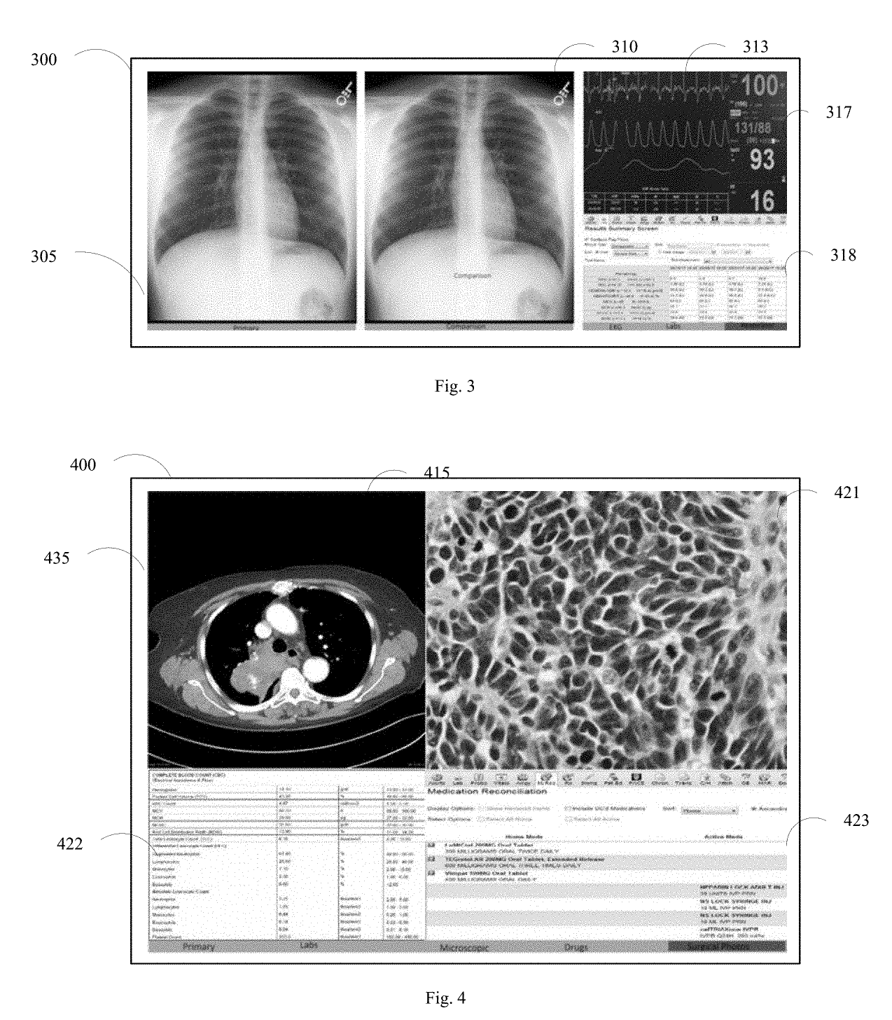

FIG. 3 illustrates exemplary panel placement with extrinsic logical panels on a monitor configuration, in accordance with an embodiment of the present invention. Internists and other professionals caring for patients in an intensive care unit (ICU) may regularly review imaging studies, and also may need to view and interact with data that is extrinsic to the imaging environment, such as, but not limited to, patient vital sign monitors, laboratory data, respirators, etc. The VRC may support these extrinsic logical panels, allowing their automatic placement and integration with the other imaging panels. In some embodiments, the VRC may obtain these data from external sources and plot them on appropriate panels. In other embodiments, the VRC may simply provide a window and its logical handles to the external software to allow that software to plot the data and manage its interaction directly. In the present embodiment, an internist may use a three monitor configuration 300 in the ICU. Configuration 300 may include a first vertical monitor 305, a second vertical monitor 310, and a third vertical monitor 313. In the present embodiment, the primary chest film may be displayed on monitor 305. The comparison chest film may be displayed on monitor 310. Monitor 313 may display two logical panels such as, but not limited to, Vitals (EKG and other live patient data) 317 and Laboratory (test results) 318. Two other panels such as, but not limited to, Patient Orders and Clinical Notes may also be mapped to the third monitor, selectable but not displayed by default. Other embodiments may use various other extrinsic panels. In some alternate embodiments, the VRC may support remote viewing of the displays.

FIG. 4 illustrates exemplary panel placement with patient data panels on a monitor configuration, in accordance with an embodiment of the present invention. In a non-limiting example, at a multispecialty tumor conference, a pathologist may presents cases to medical specialists using a single large screen television as a display. In contrast to the radiologist, who typically focuses on viewing primary and comparison imaging studies, the pathologist wishes to show the primary imaging exam alongside his microscopic slides of the tumor. In addition, a separate panel with clinical data about the patient and his medical background may be also provided. In the present embodiment, configuration 400 may include a large screen single monitor 435. On monitor 435 a primary 415, a microscopic image 421, blood count 422, and medical reconciliation 423 may be displayed. In the present embodiment, this placement of panels may be different than what the pathologist typically uses in his office, but this may not violate the display independent approach to the functional panels. After all, he is using a large single panoramic display to present to an audience, rather than one or two smaller monitors in his office. It may be natural for him to configure that display specifically for this presentation rather than his usual workflow. Even so, the functionality of the individual panels may be still defined by the same display independent parameters as those used on his office displays or even his tablet computer.

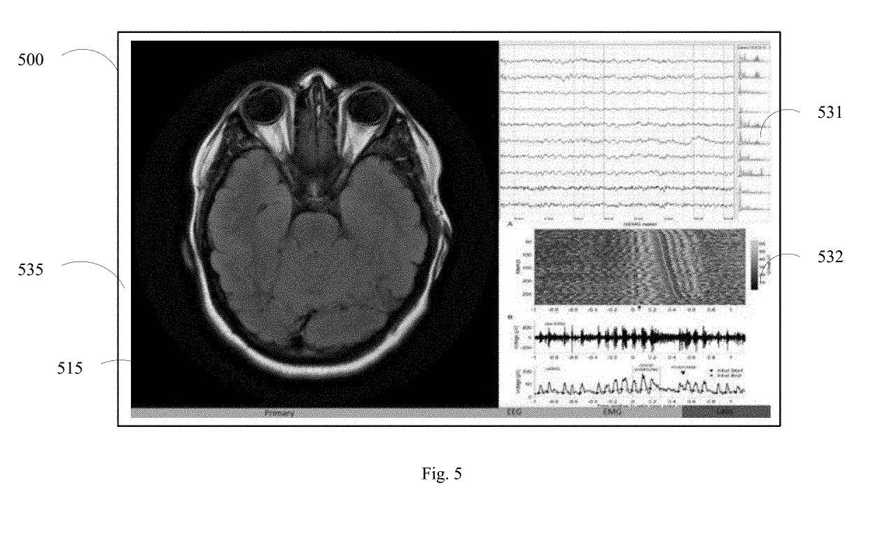

FIG. 5 illustrates exemplary panel placement with patient data panels on a monitor configuration, in accordance with an embodiment of the present invention. In a non-limiting example, Neurologists may use medical images, especially MRI, but also other clinical studies such as, but not limited to, electroencephalograms (EEGs), electromyograms (EMGs), sleep and laboratory studies, etc. As a neurologist may be dictating his notes and reports on a patient, he may use a larger array of functional panels than a radiologist, but still wants to interact with them in a familiar way, divorced from the details of physical displays. In the present embodiment, configuration 500 may include a single monitor 535. On monitor 535 a primary 515, an EEG image 531, and an EMG 532 may be displayed.

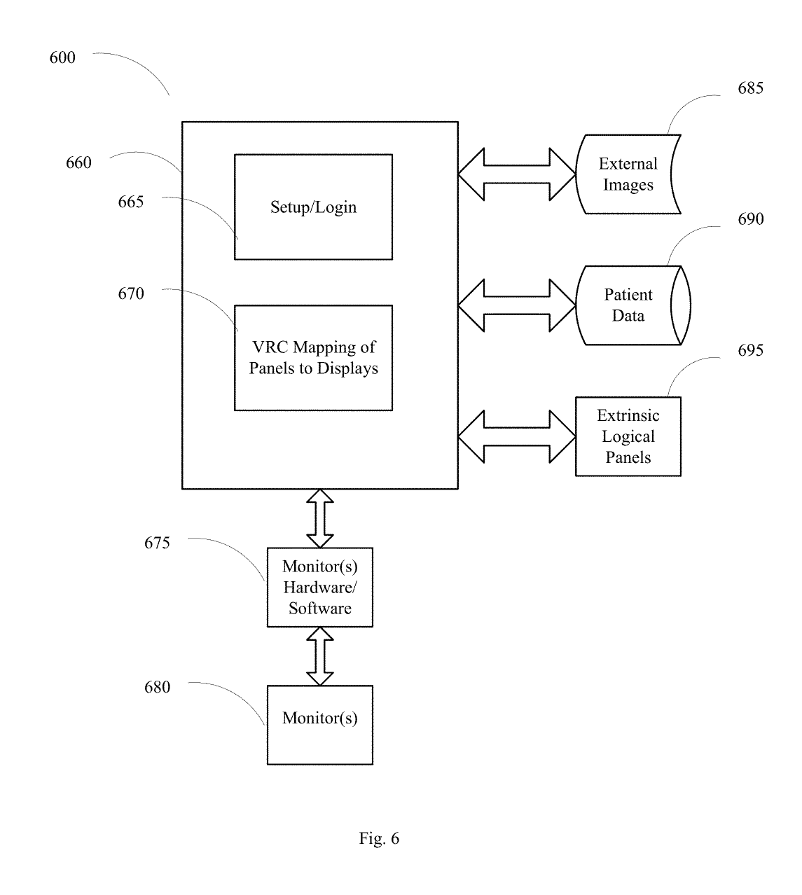

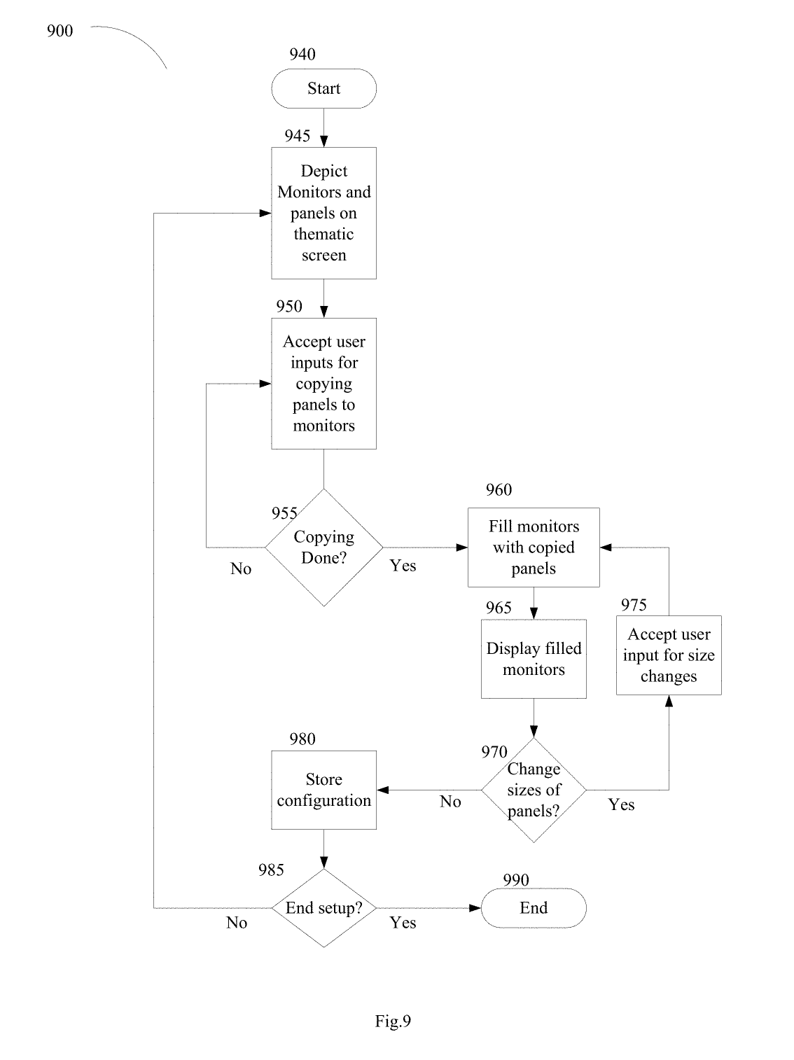

FIG. 6 illustrates an exemplary VRC system, in accordance with an embodiment of the present invention. In the present embodiment, a system 600 may include a VRC program 660, an external image database 685, a patient database 690, extrinsic logical panels 695, monitor(s) hardware/software 675, and monitor(s) 680. VRC program 660 may include a setup/login component 665, and a VRC mapping component 670. VRC program 660 may communicate bi-directionally with external image database 685, patient database 690, extrinsic logical panels 695, and monitor(s) hardware/software 675. Monitor(s) hardware/software 675 may communicate bi-directionally with monitor(s) 680. VRC program 660 may reside on various computing systems such as, but not limited to, exam viewing stations, personal computers, laptops, tablets, smartphones, etc. External image database 685 may include any images/data that may be stored on a remote storage system such as, but not limited to, PACS, cloud storage, server system on remote network, etc. Patient database 690 may include any local storage on a local network. Extrinsic logical panels 695 may include any local data sources such as, but not limited to, patient vital sign monitors, laboratory data, respirators, imaging acquisition or manipulation stations such as, but not limited to, CT/MRI image manipulation/reconstruction computers, etc. In some alternate embodiments, remote data sources may communicate data in essentially real-time. In the present embodiment, VRC mapping component 670 may manage the details of mapping the functional panels onto whatever physical display(s) the radiologist may be currently using. VRC mapping component 670 may communicate with Monitor(s) hardware/software 675 with mapped displays for display on monitor(s) 680 according to preferences of the user. In some alternate embodiments, VRC program 660 may reside on a removable media such as, but not limited to, a DVD, a CD, a flash drive, etc. along with selected images and or data. In some other alternate embodiments, the removable media may also include a GUI setup program 900 such as, without limitation, as described in FIG. 9. In some other embodiments, the VRC program 600 and/or GUI setup program 900 may be auto-executing upon mounting the removable media to a computing device.

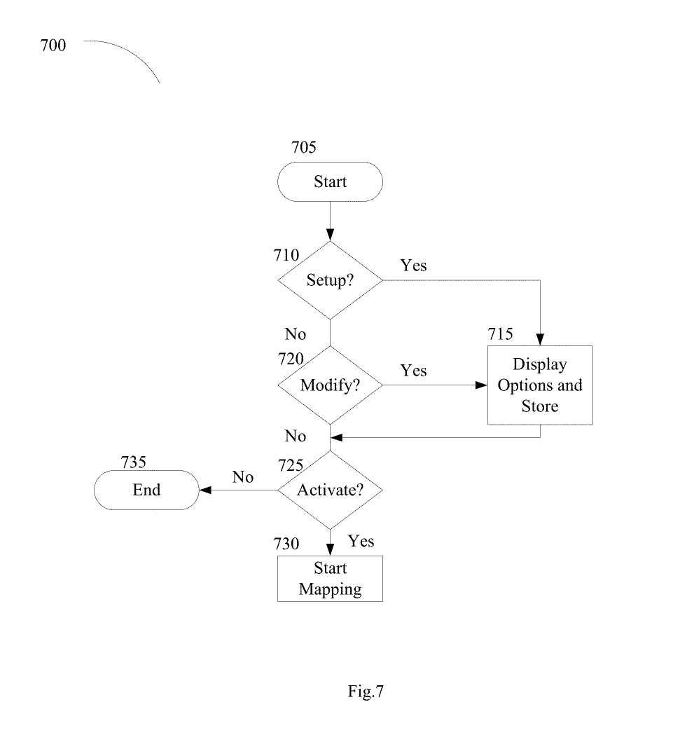

FIG. 7 illustrates an exemplary setup method, in accordance with an embodiment of the present invention. In the present embodiment, a process 700 may start at a step 705 where the user may login. In a step 710 it may be determined if the user has a completed preferences about how to use functional panels. If the user may have not completed entering preferences, the process may proceed to a step 715 where options for setting preferences may be displayed to the user and the user's chosen options may be saved. Upon storing the user's preferences, the process may return to a step 725. If the user has completed preferences, the process may proceed from step 710 to a step 720. In step 720 it may be determined if the user desires to modify stored preferences. If the user may desire to modify preferences, the process may proceed to step 715. If the user may not desire modifications the process may proceed to step 725. In step 725 it may be determined if the user desires to start a viewing session. If the user may desire to start a session the process may proceed to a step 730 where a VRC mapping process may start. If the user may not desire to start a session, the process may end at a step 735.

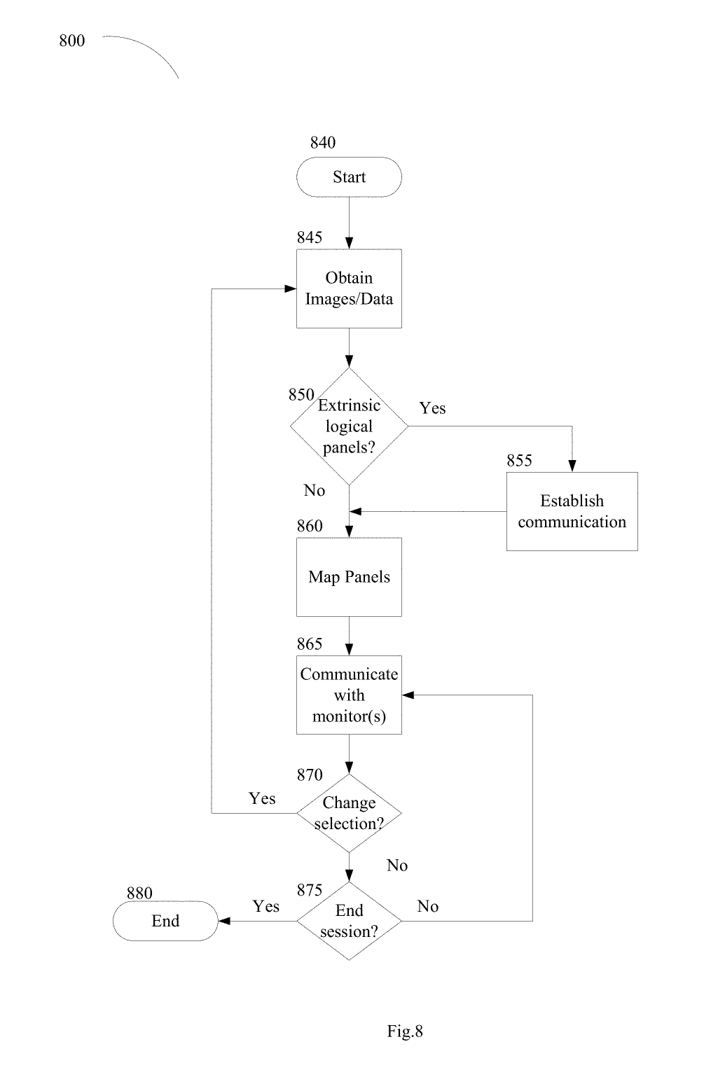

FIG. 8 illustrates an exemplary mapping method, in accordance with an embodiment of the present invention. In the present embodiment, a process 800 may start at a step 840 where the user may desire to start a viewing session. In a step 845 required data for the session may be retrieved from databases 685 and/or 690. In a step 850 it may be determined if extrinsic logical panels 695 may be required. If extrinsic logical panels 695 may be required, the process proceeds to a step 855 where communications with local devices may be established. In some alternative embodiments, communications may be established with remote devices. If extrinsic logical panels 695 may not be required, the process proceeds to a step 860. In step 860 the VRC maps 670 the images/data obtained to the locally attached monitor(s) according to the preferences of the user. In a step 865 this mapping may then be communicated to the local hardware/software 675 for display on the monitor(s) 680. In some alternative embodiments, the mapping may be communicated to remote hardware/software 675 for display on remote monitor(s) 680. The process may then proceed to a step 870 where it is determined if the user requests a selection change for the display. If a selection change is desired, the process returns to step 845 to obtain any images/data that may be required for the change. If changes may not be desired, the process may proceed to a step 875. In step 875 it may be determined if the user desired to end the session. If the user may not desire to end the process returns to step 865. If the user desires to end the session, the process may proceed to a step 880 to end the session. In some alternate embodiments, the session may be recorded or stored for, but not limited to, educational purposed.

FIG. 9 illustrates an exemplary GUI method, in accordance with an embodiment of the present invention. In the present embodiments, display profiles may allow the radiologist to specify the relational position of panels in different display configurations. These relational positions may be stored as attributes to allow positioning of panels on similar display configurations encountered in the future. In the present embodiment, a process 900 may start at a step 940 where the user may desire to start a setup session. In a step 945 the radiologist may be presented with a thematic screen depicting a selected amount of monitors and a selected amount of panels. In some embodiments, the monitors and the panels are representations. In some embodiments, the selected amounts may be determined by the user. In some embodiments, the selected amounts may be determined by the method. In a step 950 the method may accept may user inputs for copying a selected panel to a location inside a monitor. In the present embodiment, the user may utilize various processes to copy the panel to the monitor such as, but not limited to, click and drag with a mouse, touch and drag on a touch panel, keyboard entry, etc. The radiologist may not need to place these panels precisely in the symbolic monitors since their size and dimension is not known. In a step 955 it may be determined if the user has completed copying the desired panels to the monitors. The radiologist may click on a “Done” button to indicate the completion. In the present embodiment, the “Done” button may be an area on screen 100, a keyboard entry, a mouse control, a touch control, etc. If the radiologist has indicated “Done”, the method proceeds to a step 960 where the monitors are filled with the copied panels according to stored fill parameters and then displayed to the user in a step 965. In the present embodiment, a default fill parameter may be equal percentages. In a non-limiting example, if two panels are to be filled on one monitor, each panel may fill 50% of the monitor area. In other embodiments, the fill parameters may be set by the user before starting the GUI method. In a step 970, it may be determined if the user desires to change the relative sizes of the displayed panels on the monitors. If it is determined that the user wishes to change the sizes, the method may proceed to a step 975 where inputs may be accepted from the user. The user inputs may include, but not limited to, a keyboard entry, a mouse control, a touch control, etc. The radiologist may click on a “Done” button to indicate the completion. In the present embodiment, the “Done” button may be an area on screen 100, a keyboard entry, a mouse control, a touch control, etc. If the radiologist has indicated “Done”, the method proceeds to a step 980 where the current configuration is stored for the user. In some embodiments the configuration is stored as a display list. The process may then proceed to a step 985 where it is determined if the setup method may be completed. If the setup has completed, the process may proceed to a step 990 to end the. If the setup has not completed the method may return to step 945 where the radiologist may be presented with a thematic screen depicting a selected amount of monitors and a selected amount of panels. In some embodiments, the selected amounts may be determined by the user. In some embodiments, the selected amounts may be determined by the method.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

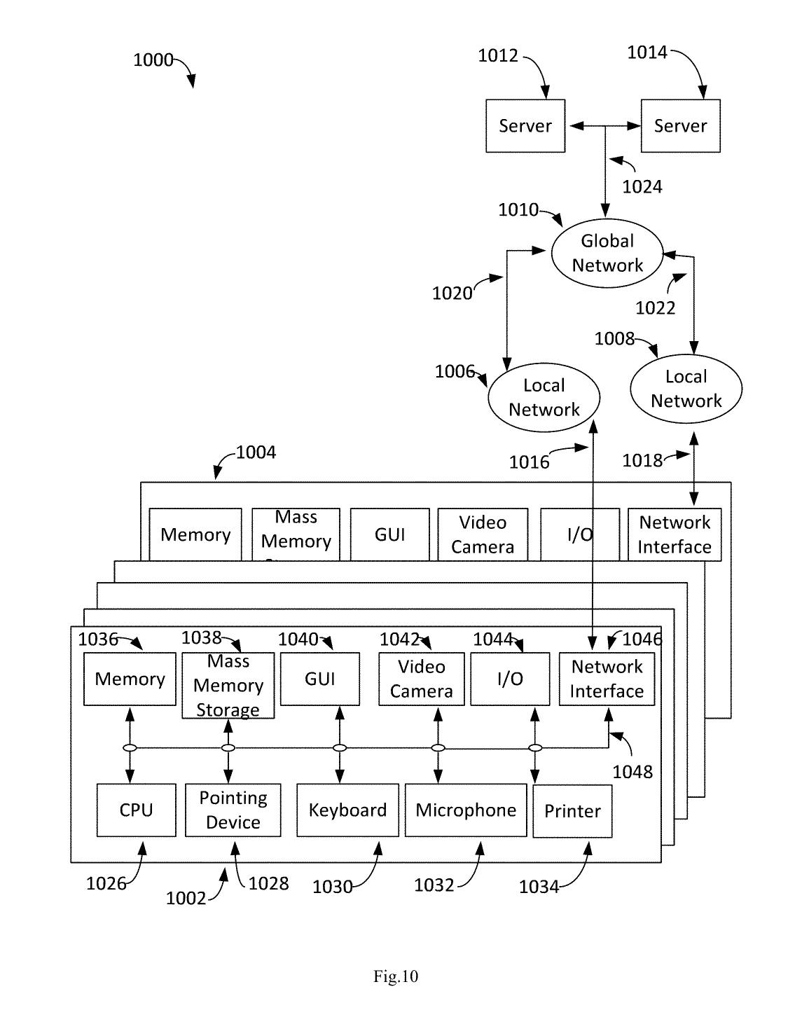

FIG. 10 is a block diagram depicting an exemplary client/server system which may be used by an exemplary web-enabled/networked embodiment of the present invention.

A communication system 1000 includes a multiplicity of clients with a sampling of clients denoted as a client 1002 and a client 1004, a multiplicity of local networks with a sampling of networks denoted as a local network 1006 and a local network 1008, a global network 1010 and a multiplicity of servers with a sampling of servers denoted as a server 1012 and a server 1014.

Client 1002 may communicate bi-directionally with local network 1006 via a communication channel 1016. Client 1004 may communicate bi-directionally with local network 1008 via a communication channel 1018. Local network 1006 may communicate bi-directionally with global network 1010 via a communication channel 1020. Local network 1008 may communicate bi-directionally with global network 1010 via a communication channel 1022. Global network 1010 may communicate bi-directionally with server 1012 and server 1014 via a communication channel 1024. Server 1012 and server 1014 may communicate bi-directionally with each other via communication channel 1024. Furthermore, clients 1002, 1004, local networks 1006, 1008, global network 1010 and servers 1012, 1014 may each communicate bi-directionally with each other.

In one embodiment, global network 1010 may operate as the Internet. It will be understood by those skilled in the art that communication system 1000 may take many different forms. Non-limiting examples of forms for communication system 1000 include local area networks (LANs), wide area networks (WANs), wired telephone networks, wireless networks, or any other network supporting data communication between respective entities.

Clients 1002 and 1004 may take many different forms. Non-limiting examples of clients 1002 and 1004 include personal computers, personal digital assistants (PDAs), cellular phones and smartphones.

Client 1002 includes a CPU 1026, a pointing device 1028, a keyboard 1030, a microphone 1032, a printer 1034, a memory 1036, a mass memory storage 1038, a GUI 1040, a video camera 1042, an input/output interface 1044, and a network interface 1046.

CPU 1026, pointing device 1028, keyboard 1030, microphone 1032, printer 1034, memory 1036, mass memory storage 1038, GUI 1040, video camera 1042, input/output interface 1044 and network interface 1046 may communicate in a unidirectional manner or a bi-directional manner with each other via a communication channel 1048. Communication channel 1048 may be configured as a single communication channel or a multiplicity of communication channels.

CPU 1026 may be comprised of a single processor or multiple processors. CPU 1026 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general purpose microprocessors.

As is well known in the art, memory 1036 is used typically to transfer data and instructions to CPU 1026 in a bi-directional manner. Memory 1036, as discussed previously, may include any suitable computer-readable media, intended for data storage, such as those described above excluding any wired or wireless transmissions unless specifically noted. Mass memory storage 1038 may also be coupled bi-directionally to CPU 1026 and provides additional data storage capacity and may include any of the computer-readable media described above. Mass memory storage 1038 may be used to store programs, data and the like and is typically a secondary storage medium such as a hard disk. It will be appreciated that the information retained within mass memory storage 1038, may, in appropriate cases, be incorporated in standard fashion as part of memory 1036 as virtual memory.

CPU 1026 may be coupled to GUI 1040. GUI 1040 enables a user to view the operation of computer operating system and software. CPU 1026 may be coupled to pointing device 1028. Non-limiting examples of pointing device 1028 include computer mouse, trackball and touchpad. Pointing device 1028 enables a user with the capability to maneuver a computer cursor about the viewing area of GUI 1040 and select areas or features in the viewing area of GUI 1040. CPU 1026 may be coupled to keyboard 1030. Keyboard 1030 enables a user with the capability to input alphanumeric textual information to CPU 1026. CPU 1026 may be coupled to microphone 1032. Microphone 1032 enables audio produced by a user to be recorded, processed and communicated by CPU 1026. CPU 1026 may be connected to printer 1034. Printer 1034 enables a user with the capability to print information to a sheet of paper. CPU 1026 may be connected to video camera 1042. Video camera 1042 enables video produced or captured by user to be recorded, processed and communicated by CPU 1026.

CPU 1026 may also be coupled to input/output interface 1044 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 1026 optionally may be coupled to network interface 1046 which enables communication with an external device such as a database or a computer or telecommunications or internet network using an external connection shown generally as communication channel 1016, which may be implemented as a hardwired or wireless communications link using suitable conventional technologies. With such a connection, CPU 1026 might receive information from the network, or might output information to a network in the course of performing the method steps described in the teachings of the present invention.

In some embodiments, the teachings of the present invention may be applied by, but not limited to, internal medicine physicians in the ICU, pathologists at a case conference, neurologist integrating his imaging and medical information on the patient, etc. In some non-limiting examples, a surgeon working on a patient preparing for surgery, a rehabilitation physician using integrated imaging and medical data to care for a patient, etc. may utilize the teachings of the present invention. In some embodiments, the teachings of the present invention may be applied by, but not limited to, a manufacturer creating preference sets which may be published and provided to radiologists without the need for much custom configuration.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention. Thus, some alternate embodiments of the present invention may be configured to comprise a smaller subset of the foregoing means for and/or steps described that the applications designer will selectively decide, depending upon the practical considerations of the particular implementation, to carry out and/or locate within the jurisdiction of the USA. For example, any of the foregoing described method steps and/or system components which may be performed remotely over a network (e.g., without limitation, a remotely located server) may be performed and/or located outside of the jurisdiction of the USA while the remaining method steps and/or system components (e.g., without limitation, a locally located client) of the forgoing embodiments are typically required to be located/performed in the USA for practical considerations. In client-server architectures, a remotely located server typically generates and transmits required information to a US based client, for use according to the teachings of the present invention. Depending upon the needs of the particular application, it will be readily apparent to those skilled in the art, in light of the teachings of the present invention, which aspects of the present invention can or should be located locally and which can or should be located remotely. Thus, for any claims construction of the following claim limitations that are construed under 35 USC § 112 (6) it is intended that the corresponding means for and/or steps for carrying out the claimed function are the ones that are locally implemented within the jurisdiction of the USA, while the remaining aspect(s) performed or located remotely outside the USA are not intended to be construed under 35 USC § 112 (6). In some embodiments, the methods and/or system components which may be located and/or performed remotely include, without limitation, remote viewing.

It is noted that according to USA law, all claims must be set forth as a coherent, cooperating set of limitations that work in functional combination to achieve a useful result as a whole. Accordingly, for any claim having functional limitations interpreted under 35 USC § 112 (6) where the embodiment in question is implemented as a client-server system with a remote server located outside of the USA, each such recited function is intended to mean the function of combining, in a logical manner, the information of that claim limitation with at least one other limitation of the claim. For example, in client-server systems where certain information claimed under 35 USC § 112 (6) is/(are) dependent on one or more remote servers located outside the USA, it is intended that each such recited function under 35 USC § 112 (6) is to be interpreted as the function of the local system receiving the remotely generated information required by a locally implemented claim limitation, wherein the structures and or steps which enable, and breathe life into the expression of such functions claimed under 35 USC § 112 (6) are the corresponding steps and/or means located within the jurisdiction of the USA that receive and deliver that information to the client (e.g., without limitation, client-side processing and transmission networks in the USA). When this application is prosecuted or patented under a jurisdiction other than the USA, then “USA” in the foregoing should be replaced with the pertinent country or countries or legal organization(s) having enforceable patent infringement jurisdiction over the present application, and “35 USC § 112 (6)” should be replaced with the closest corresponding statute in the patent laws of such pertinent country or countries or legal organization(s).

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” or “steps for” claim limitation implies that the broadest initial search on 35 USC § 112(6) (post AIA 112(f)) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112(6) (post AIA 112(f)) when such corresponding structures are not explicitly disclosed in the foregoing patent specification. Therefore, for any invention element(s)/structure(s) corresponding to functional claim limitation(s), in the below claims interpreted under 35 USC § 112(6) (post AIA 112(f)), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC § 112(6) (post AIA 112(f)) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112(6) (post AIA 112(f)), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of mapping of functional panels onto available physical displays according to the present invention will be apparent to those skilled in the art. Various aspects of the invention have been described above by way of illustration, and the specific embodiments disclosed are not intended to limit the invention to the particular forms disclosed. The particular implementation of the mapping of functional panels onto available physical displays may vary depending upon the particular context or application. By way of example, and not limitation, the mapping of functional panels onto available physical displays described in the foregoing were principally directed to mapping medical images onto imaging stations implementations; however, similar techniques may instead be applied to mapping medical images onto VR displays, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

What is claimed is:

1. A method comprising the steps of:
retrieving session data for a radiologist, the session data at least comprising viewing preferences for the radiologist, the radiologist at least utilizing a computing device for viewing images for a session;
accessing at least two functional panels associated with the session;
mapping the at least two functional panels to display devices in communication with the computing device, said mapping at least using the viewing preference and configurations of the display devices; and
communicating said mapping to the display devices for viewing by the radiologist.

2. The method as recited in claim 1, further comprising:
accessing at least one extrinsic logical panel; and
including the at least one extrinsic logical panel in said mapping.

3. The method as recited in claim 1, further comprising receiving a selection for the session from the radiologist.

4. The method as recited in claim 1, further comprising activating a setup method for obtaining the viewing preferences.

5. The method as recited in claim 1, in which the at least two functional panels comprises at least a primary panel and a comparison panel.

6. The method as recited in claim 1, in which the viewing preferences comprises at least one display list comprising at least an order for panel display.

7. The method as recited in claim 5, in which the at least two functional panels further comprises a selection panel comprising sessions available for viewing.

8. The method as recited in claim 7, in which the radiologist selects a session from said selection panel.

9. The method as recited in claim 1, in which the session data further comprises panel attributes for use during said mapping.

10. The method as recited in claim 9, in which the panel attributes comprise relational attributes and intrinsic attributes.

11. A method comprising:
steps for retrieving session data for a radiologist;
steps for accessing at least two functional panels associated with a session;
steps for mapping the at least two functional panels to display devices; and
steps for communicating said mapping to the display devices for viewing by the radiologist.

12. The method as recited in claim 11, further comprising:
steps for accessing at least one extrinsic logical panel; and
steps for including the at least one extrinsic logical panel in said mapping.

13. The method as recited in claim 11, further comprising steps for receiving a selection for the session from the radiologist.

14. The method as recited in claim 11, further comprising steps for activating a setup method for obtaining the viewing preferences.

15. A method comprising the steps of:
depicting at least a selected amount of monitors and a selected amount of panels on a thematic screen;
accepting user inputs for copying at least one panel to at least one monitor;
filling the monitors with the copied panels;
displaying the filled monitors to the user; and
storing a configuration for the filled monitors as a viewing preference.

16. The method as recited in claim 15, further comprising accepting inputs from the user for changing relative sizes of the panels.

17. The method as recited in claim 15, in which said filing uses stored fill parameters.

18. The method as recited in claim 15, in which the configuration at least comprises a display list.

19. A method comprising:
steps for depicting a selected amount of monitors and a selected amount of panels on a thematic screen;
steps for accepting user inputs for copying one panel to one monitor;
steps for filling the monitors with the copied panels;
steps for displaying the filled monitors to the user; and
steps for storing a configuration for the filled monitors as a viewing preference.

20. The method as recited in claim 19, further comprising steps for accepting inputs from the user for changing relative sizes of the panels.

21. A non-transitory computer-readable storage medium with an executable program stored thereon, wherein the program instructs one or more processors to perform the following steps:
retrieving session data for a radiologist, the session data at least comprising viewing preferences for the radiologist, the radiologist at least utilizing a computing device for viewing images for a session;
accessing at least two functional panels associated with the session;
mapping the at least two functional panels to display devices in communication with the computing device, said mapping at least using the viewing preference and configurations of the display devices; and
communicating said mapping to the display devices for viewing by the radiologist.

22. The program instructing the one or more processors as recited in claim 21, further comprising:
accessing at least one extrinsic logical panel; and
including the at least one extrinsic logical panel in said mapping.

23. The program instructing the one or more processors as recited in claim 21, further comprising receiving a selection for the session from the radiologist.

24. The program instructing the one or more processors as recited in claim 21, further comprising activating a setup method for obtaining the viewing preferences.

25. The program instructing the one or more processors as recited in claim 21, in which the at least two functional panels comprises at least a primary panel and a comparison panel.

26. The program instructing the one or more processors as recited in claim 21, in which the viewing preferences comprises at least one display list comprising at least an order for panel display.

27. The program instructing the one or more processors as recited in claim 25, in which the at least two functional panels further comprises a selection panel comprising sessions available for viewing.

28. The program instructing the one or more processors as recited in claim 27, in which the radiologist selects a session from said selection panel.

29. The program instructing the one or more processors as recited in claim 21, in which the session data further comprises panel attributes for use during said mapping.

30. The program instructing the one or more processors as recited in claim 29, in which the panel attributes comprise relational attributes and intrinsic attributes.
